(19) United States
(12) Patent Application Publication
(10) Pub. No.: US 2025/0259737 A1
(43) Pub. Date: Aug. 14, 2025

(54) MEDICAL SERVICE SUPPORT DEVICE, METHOD, AND PROGRAM

(71) Applicant: FUJIFILM CORPORATION, Tokyo (JP)

(72) Inventor: Kota KAWAGUCHI, Tokyo (JP)

(21) Appl. No.: 19/043,530

(22) Filed: Feb. 3, 2025

(30) Foreign Application Priority Data
Feb. 13, 2024 (JP) 2024-019652

(51) Int. Cl. G16H 40/20

(52) U.S. Cl. CPC G16H 40/20

(57) ABSTRACT

A processor is configured to acquire action information related to an action of a medical worker, classify the action information into a plurality of groups based on attribute information related to classification, derive numerical information related to the action of the medical worker and the number of medical workers based on the action information for each of the plurality of groups, and derive a representation result in which the numerical information and the number of medical workers are represented in a unit of the classified group.

### CROSS REFERENCE TO RELATED APPLICATIONS

The present application claims priority from Japanese Patent Application No. 2024-019652, filed on Feb. 13, 2024, the entire disclosure of which is incorporated herein by reference.

### BACKGROUND

### Technical Field

The present disclosure relates to a medical service support device, a method, and a program.

### Related Art

In a medical institution, various types of orders are issued, and various medical practices are performed according to the orders each time. For example, JP2008-282403A proposes managing the progress status of such a medical practice for each medical worker.

However, in the method disclosed in JP2008-282403A, it is not possible to simultaneously grasp the numerical information such as the number of cases of the actions of the medical worker in the facility such as the hospital and the number of medical workers at that time in association with each other for the administrator of the medical worker.

### SUMMARY OF THE INVENTION

The present disclosure has been made in view of the above circumstances, and an object of the present disclosure is to enable simultaneous grasping of the numerical information related to the action of the medical worker and the number of medical workers.

According to the present disclosure, there is provided a medical service support device comprising:
a processor,
in which the processor is configured to:
   acquire action information related to an action of a medical worker;
   classify the action information into a plurality of groups based on attribute information related to classification;
   derive numerical information related to the action of the medical worker and the number of medical workers based on the action information for each of the plurality of groups; and
   derive a representation result in which the numerical information and the number of medical workers are represented in a unit of the classified group.

According to the present disclosure, there is provided a medical service support method comprising:
causing a computer configured to:
   acquire action information related to an action of a medical worker;
   classify the action information into a plurality of groups based on attribute information related to classification;
   derive numerical information related to the action of the medical worker and the number of medical workers based on the action information for each of the plurality of groups; and
   derive a representation result in which the numerical information and the number of medical workers are represented in a unit of the classified group.

According to the present disclosure, there is provided a medical service support program causing a computer to execute:
a procedure of acquiring action information related to an action of a medical worker;
a procedure of classifying the action information into a plurality of groups based on attribute information related to classification;
a procedure of deriving numerical information related to the action of the medical worker and the number of medical workers based on the action information for each of the plurality of groups; and
a procedure of deriving a representation result in which the numerical information and the number of medical workers are represented in a unit of the classified group.

According to the present disclosure, it is possible to simultaneously grasp the numerical information related to the action of the medical worker and the number of medical workers.

### BRIEF DESCRIPTION OF THE DRAWINGS

FIG. 1 is a perspective view showing an outline of a medical information system to which a medical service support device according to a first embodiment of the present disclosure is applied.

FIG. 2 is a diagram showing action information.

FIG. 3 is a diagram showing a hardware configuration of the medical service support device according to the first embodiment of the present disclosure.

FIG. 4 is a diagram showing a functional configuration of the medical service support device according to the first embodiment of the present disclosure.

FIG. 5 is a diagram showing a classification result.

FIG. 6 is a diagram showing a display screen of a representation result in the first embodiment.

FIG. 7 is a diagram showing a display screen of another representation result in the first embodiment.

FIG. 8 is a flowchart showing processing performed in the first embodiment.

FIG. 9 is a diagram showing a basic region divided into regions having sizes corresponding to the number of examinations of an examination type in the second embodiment.

FIG. 10 is a diagram showing a basic region divided into regions having sizes corresponding to the number of examination items in the second embodiment.

FIG. 11 is a diagram showing a representation result in the second embodiment.

### DESCRIPTION OF THE PREFERRED EMBODIMENTS

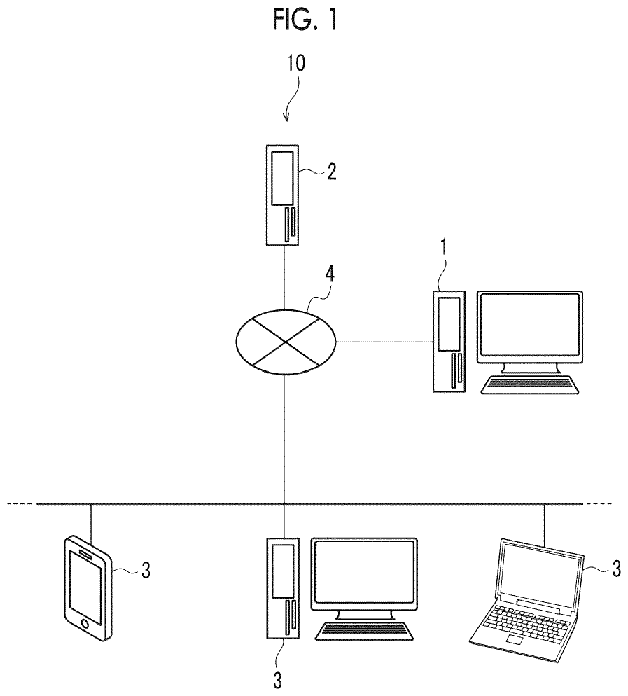

Hereinafter, embodiments of the present disclosure will be described with reference to the drawings. First, a configuration of a medical service support system to which a medical service support device according to a first embodiment of the present disclosure is applied will be described. FIG. 1 is a diagram showing a schematic configuration of a medical service support system. In a medical service support system 10 shown in FIG. 1, a medical service support apparatus 1, a management server 2, and a plurality of client terminals 3 are connected to each other via a network 4 such that they can communicate with each other. The medical service support system 10 is a system used for at least one facility (for example, a hospital) that handles a plurality of pieces of medical information.

The medical service support device 1 performs processing for supporting medical services in a hospital as described below based on the information acquired from the management server 2. A detailed configuration of the medical service support device 1 will be described later.

The management server 2 consists of a server computer or the like that manages various types of information in the hospital transmitted from the client terminal 3. The management server 2 may be a cloud server. In the present embodiment, the management server 2 manages action information related to an action of the medical worker. In the present disclosure, the medical worker is not limited to qualified persons such as a doctor, a technician, a pharmacist, and a nurse, and also includes in-hospital staff who performs reception of medical examination or inspection, guidance of patients, and the like, or unqualified persons who assist qualified persons.

The client terminal 3 is a terminal device owned by a medical worker. Examples of the client terminal 3 include a workstation, a personal computer, a tablet terminal, a smartphone, and a smart watch.

Examples of the network 4 include a wide area network (WAN). It should be noted that the WAN is merely an example, and the network 4 may be composed of at least one of a local area network (LAN), a WAN, or the like.

In the client terminal 3, information related to the actions of the medical worker is individually managed for each medical worker or each examination room, and the individual information managed for each medical worker is transmitted to the management server 2. Then, the management server 2 manages the action information related to the action of the medical worker in the hospital based on the individual action information.

In the present embodiment, as the information managed by the client terminal 3, for example, in a case of the client terminal 3 of a doctor, information such as a doctor name, a medical department, a patient name, a patient gender, a patient date of birth, a medical examination date and time, a disease name, an examination content, an examination result, a diagnosis content, a treatment content, a treatment result, a content of surgery, a result of surgery, whether the patient is hospitalized or outpatient, a ward of the patient in a case of hospitalization, and a reception time is included. In addition, in the case of the client terminal 3 of the examination room, information such as the examination type, the examination room, the examination device, the examination date and time, the number of technicians for each examination date and time, the patient name, the patient gender, the patient date of birth, whether the patient is hospitalized or an outpatient, the ward of the patient in the case of hospitalization, the examination reception time, the examination start time, the examination end time, the waiting time, the number of patients waiting for examination for each time slot, the requesting department, the requesting doctor, the examination reservation time, the examination reservation information (whether it is on the day or whether it is an emergency), the examination start time, the examination end time, the time when the client terminal 3 inputs the examination implementation, the time when the examination report is created, the examination state, the pathology request, and the imaging state in the case of imaging, and the like are included.

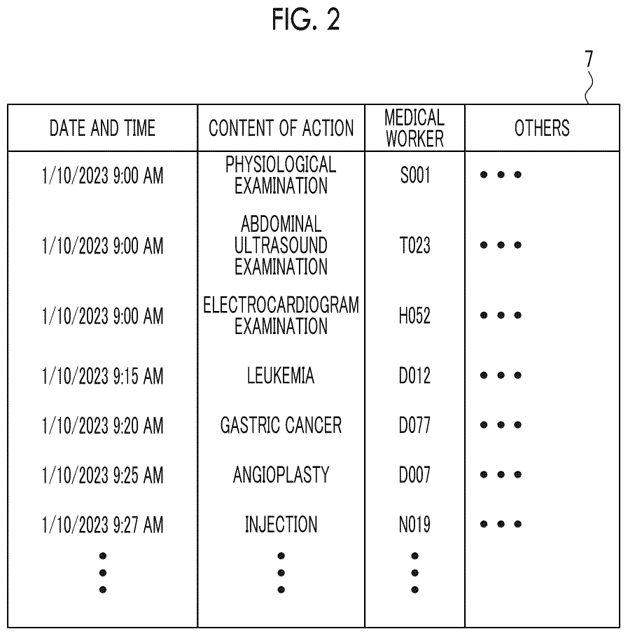

The management server 2 converts the information transmitted from the client terminal 3 into a database and manages action information related to an action of the medical worker. For example, the action information is managed by associating the action with the information transmitted from the client terminal 3. FIG. 2 is a diagram showing an example of action information managed by the management server 2. As shown in FIG. 2, the action information 7 consists of a date and time when the action of the medical worker is performed, a content of the action, the medical worker who has performed the action, and other information. Examples of the content of the action include examination, medical examination, and treatment. The examination is, for example, a physiological test, an abdominal ultrasound examination, an electrocardiogram examination, and the like. Examples of the treatment include angioplasty, medication, and injection. The medical worker is an ID number of the medical worker. The other information includes all or a part of the information transmitted from the client terminal 3.

A medical service support device 1 has a medical service support program according to the first embodiment installed therein. The medical service support device 1 may be a workstation or a personal computer installed in a hospital, or may be a server computer. The medical service support program is stored in a storage device of another server computer connected to the network or network storage (both not shown) in a state accessible from the outside, and is downloaded and installed in the medical service support device 1 in response to a request. Alternatively, the display control program is distributed in a state of being recorded on a recording medium, such as a digital versatile disc (DVD) or a compact disc read only memory (CD-ROM), and is installed in the medical service support device 1 from the recording medium.

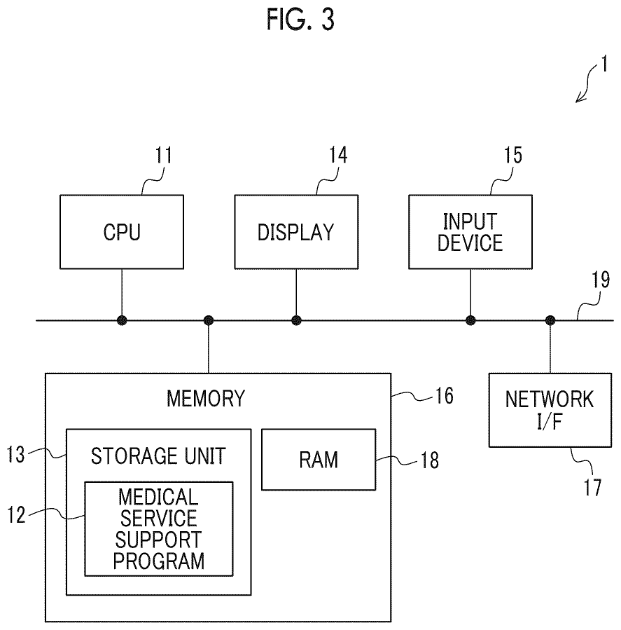

FIG. 3 is a diagram showing a hardware configuration of the medical service support device according to the first embodiment. As shown in FIG. 3, the medical service support device 1 includes a central processing unit (CPU) 11, a display 14, an input device 15, a memory 16, and a network interface (I/F) 17 connected to the network 4. The CPU 11, the display 14, the input device 15, the memory 16, and the network I/F 17 are connected to a bus 19. Note that the CPU 11 is an example of a processor according to the present disclosure.

The memory 16 includes the storage unit 13 and a random access memory (RAM) 18. The RAM 18 is a memory for primary storage and is, for example, a RAM such as a static random access memory (SRAM) or a dynamic random access memory (DRAM).

The storage unit 13 is a non-volatile memory, and is realized by at least one of, for example, a hard disk drive (HDD), a solid state drive (SSD), an electrically erasable and programmable read only memory (EEPROM), or a flash memory. The storage unit 13 as a storage medium stores the medical service support program 12 according to the present embodiment. The CPU 11 reads out the medical service support program 12 from the storage unit 13, loads the medical service support program 12 into the RAM 18, and executes the loaded medical service support program 12.

The display 14 is a device that displays various screens and is, for example, a liquid crystal display or an electro luminescence (EL) display. The input device 15 is a device for the user to provide input and is, for example, at least any of a keyboard, a mouse, a microphone for audio input, a touchpad for proximity input including a contact, or a camera for gesture input. The network I/F 17 is an interface for connecting to a network 4.

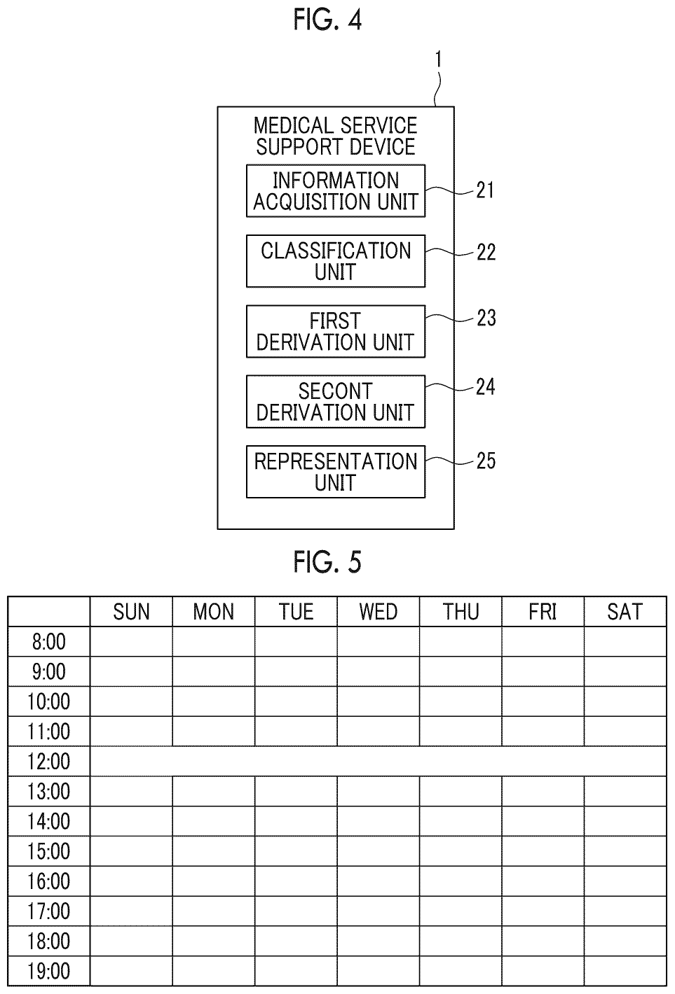

Next, a functional configuration of the medical service support device according to the first embodiment will be described. FIG. 4 is a diagram showing a functional configuration of the medical service support device according to the first embodiment. As shown in FIG. 4, the medical service support device 1 comprises an information acquisition unit 21, a classification unit 22, a first derivation unit 23, a second derivation unit 24, and a representation unit 25. Then, the CPU 11 executes the medical service support program 12, so that the CPU 11 functions as the information acquisition unit 21, the classification unit 22, the first derivation unit 23, the second derivation unit 24, and the representation unit 25.

The information acquisition unit 21 acquires action information related to the action of the medical worker from the management server 2 via the network 4 at a predetermined timing or by an instruction from an operator who is a manager of the hospital, and stores the action information in the storage unit 13. In this case, the action information of all the dates and times managed by the management server 2 may be acquired, or the action information for the designated period may be acquired. Examples of the method of designating the period include designation by a start date and time and an end date and time, such as Jan. 1, 2023 to Dec. 31, 2023. In addition, for example, a designation based on the number of days from a certain date, such as 180 days from Jan. 1, 2023, can be mentioned.

The classification unit 22 classifies the action information into a plurality of groups based on the attribute information related to the classification. In the first embodiment, it is assumed that a physiological examination is used as the action and two pieces of attribute information of a day of the week and a time slot are used as the attribute information. The day of the week is an example of first attribute information of the present disclosure, and the time slot is an example of second attribute information of the present disclosure. The attribute information may be predetermined by the medical service support program, but the classification unit 22 may acquire the designated attribute information from the plurality of pieces of attribute information and classify the action information into a plurality of groups based on the acquired attribute information.

In the first embodiment, the day of the week is set as an initial value of the attribute information, and the time slot is fixed to the hospital's medical care time. In addition, regarding the day of the week, the operator can designate attribute information other than the day of the week using the input device 15 as will be described later. Examples of the attribute information other than the day of the week include at least one of an action performance date, a medical worker who performed the action, a room in which the action was performed, a device used for the action, or an action type. In the first embodiment, since the action is a physiological examination, for example, the technician who performed the examination, the type of the physiological examination, the examination room, and the examination date can be used as attribute information other than the day of the week. In this case, the classification unit 22 classifies the action information into a plurality of groups based on the designated attribute information based on the designated attribute information.

The day of the week is seven days from Sunday to Saturday. The time slot is obtained by dividing the medical care time during which the examination can be performed at the hospital in units of one hour. For example, in a case where the start time of the examination in the hospital is 8:00 and the end time of the examination is 19:00, 11 time slots excluding the lunch break (12:00 to 13:00) are acquired. Therefore, as shown in FIG. 5, the action information is classified into a total of 77 groups according to seven days of the week and eleven time slots. In FIG. 5, the groups are classified as a matrix in which a horizontal axis to which a day of the week is assigned and a vertical axis to which a time slot is assigned are defined and cells corresponding to the groups are two-dimensionally arranged.

The first derivation unit 23 derives the numerical information and the number of medical workers related to the action of the medical worker based on the action information for each of the plurality of groups. That is, the numerical information and the number of the medical workers related to the action of the medical worker in each of the 11 time slots from 8:00 to 19:00 of each day of the week are derived. In the first embodiment, the number of the physiological examinations is set as the initial value of the numerical information. In this case, the first derivation unit 23 selects the action information of which the content of the action is “physiological examination” from all the action information acquired by the information acquisition unit 21 and stored in the storage unit 13. Then, the number of examinations and the number of medical workers are derived based on the selected action information.

The number of medical workers may be weighted according to factors such as the length of service or qualifications held by the medical workers. For example, for the resident and the fellow, the weighting may be performed such that the weighting for the resident is a relatively low value as compared with the fellow, for example, the weighting for the resident is 0.8 and the weighting for the fellow is 1.0. In addition, weighting may be performed according to the number of experiences of the medical worker for the examination type or the examination item (substantially the same as the number of examinations) or the private circumstances (the performance is lowered because the medical worker has just returned to work, the performance is lowered due to a disability or an injury, and the like).

In addition, the number of examinations may also be weighted according to the difficulty of the examination. For example, weighting may be performed such that the relatively difficult examination has a relatively high value. The difficulty of the examination may be determined based on the total number of examinations performed at the facility or qualifications required for the examination. In addition, the difficulty of the examination may be determined according to the number of cases (substantially the same as the number of examinations) viewed in a specific range such as a facility average, a group average, or a national average (fewer cases indicate less familiarity), the re-examination rate (a higher rate of re-examination after failure indicates greater difficulty), the qualification possession rate in the facility, the degree of difficulty set in the facility, or the classification determined by the Ministry of Health, Labour and Welfare (https://www.mhlw.go.jp/stf/shingi/2r9852000002i332-att/2r9852000002jcdo.pdf). In addition, the weighting value may be a set value or a variable value that varies according to an input of the user.

In the first embodiment, as will be described later, the operator can designate the numerical information other than the number of examinations by using the input device 15. As the numerical information other than the number of examinations, the number of technicians who perform the examination, a technician operation rate, a patient waiting time, the number of patients waiting for the examination, and a reservation/examination deviation time can be used.

The technician operation rate can be calculated by “standard operation time #(standard operation time+standard operation interval)” using standard operation time indicating a length of an operation time of the technician and a standard operation interval indicating an interval before and after operation of the technician, in a corresponding time slot of a corresponding day of the week.

The patient waiting time is a time from the arrival of the patient in the examination room to the start of the examination in the corresponding time slot on the corresponding day of the week. The number of patients waiting for the examination is a statistical value of patients who are waiting for examination in a corresponding time slot on a corresponding day of the week. As the statistical value, for example, a maximum value, an average value, a median value, or the like can be used, but the present invention is not limited thereto. The reservation/examination deviation time is a difference between the examination reservation time of the patient and the time at which the examination actually starts in a corresponding time slot on a corresponding day of the week.

In the first embodiment, the first derivation unit 23 classifies the number of examinations into, for example, four ranges by the threshold value processing. The first derivation unit 23 classifies the number of examinations into, for example, four ranges of 0 to 10, 11 to 20, 21 to 30, and 31 or more. The classification of the number of examinations is not limited to four, and may be two, three, or five or more. In addition, the first derivation unit 23 classifies the number of medical workers into, for example, three ranges in the threshold value processing. For example, the number of medical workers is classified into three ranges of 0 to 4, 5 to 9, and 10 or more. The classification of the number of medical workers is not limited to three, and may be two or four or more.

The second derivation unit 24 derives the workload in each of the plurality of groups based on at least the numerical information. The workload may be used as the numerical information itself, but in the first embodiment, a value obtained by dividing the number of examinations by the number of medical workers is derived as the workload. The workload is greater as the number of examinations is greater or the number of medical workers is smaller.

In the first embodiment, the second derivation unit 24 classifies the derived workload by threshold value processing. For example, the degree of activity is classified into a workload equal to or higher than a threshold value Th1 and a workload lower than the threshold value Th1. The workload, using two threshold values Th2 and Th3 (Th2>Th3), may be classified into three ranges of a workload equal to or greater than the threshold value Th2, a workload equal to or greater than the threshold value Th3 and less than the threshold value Th2, and a workload less than the threshold value Th3. Further, the workload may be classified into four or more ranges using three or more threshold values.

The threshold value can be arbitrarily set by the operator. A threshold value may be set using a statistical value of the past workload of the hospital. As the statistical value, a minimum value, a maximum value, an average value, a variance value, and the like can be used. Alternatively, the threshold value may be set using a statistical value of past workloads of a plurality of hospitals existing in a region or hospitals in the whole country.

The workload may be derived as the operation rate of the medical worker. The operation rate of the medical worker can be calculated by “standard operation time: (standard operation time+standard operation interval)” using standard operation time indicating a length of operation time of the medical worker and a standard operation interval indicating an interval before and after operation of the medical worker.

The representation unit 25 derives a representation result in which the numerical information, the number of medical workers, and the workload are represented in a unit of the classified group. In the first embodiment, as shown in FIG. 5, the representation unit 25 generates a matrix in which a day of the week is assigned to a horizontal axis, a time slot is assigned to a vertical axis, and cells corresponding to the group are two-dimensionally arranged. Then, the representation unit 25 derives a representation result in which the number of examinations, the number of medical workers, and the workload are represented in a plurality of group units on the matrix. The representation unit 25 displays the representation result on the display 14. The horizontal axis is an example of the first axis of the present disclosure, and the vertical axis is an example of the second axis of the present disclosure.

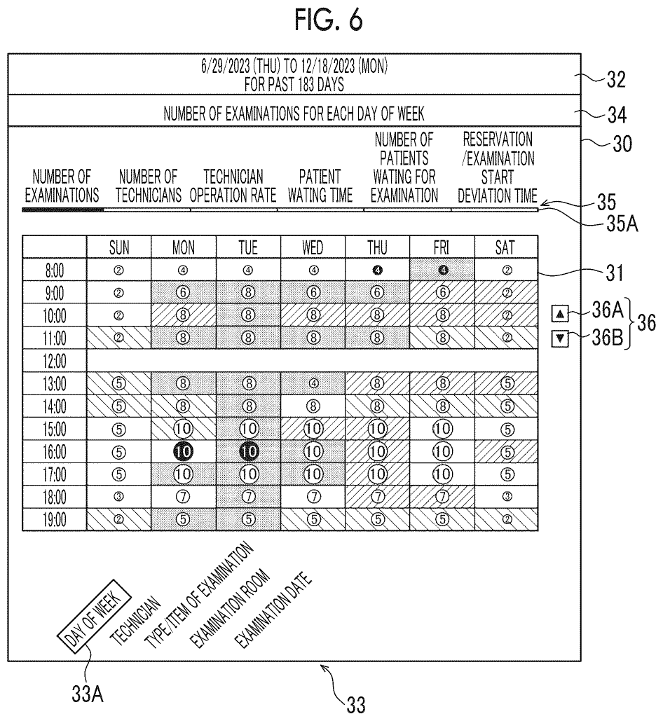

FIG. 6 is a diagram showing a display screen of a representation result in the first embodiment. As shown in FIG. 6, the display screen 30 includes a representation result 31, a period area 32, an attribute information selection area 33, a title area 34, a numerical information selection area 35, and a sort button 36.

The representation result 31 is a matrix in which a day of the week is assigned to a horizontal axis and a time slot is assigned to a vertical axis, and a plurality of groups are assigned to each cell of the matrix. In the representation result 31, the density of the color of each cell represents the number of examinations. In the present embodiment, the number of examinations is classified into four ranges as described above. Therefore, the number of examinations is represented by colors of different four stages of density. In the first embodiment, the difference in the density of the color is indicated by the application of gray to the cell and the difference in hatching. For example, the gray cell indicates that the number of examinations is large, the cell with hatching from the upper right to the lower left indicates that the number of examinations is large next to the gray cell, the cell with hatching from the upper left to the lower right indicates that the number of examinations is large next to the cell with hatching from the upper right to the lower left, and the cell with no gray and no hatching indicates that the number of examinations is the smallest.

In addition, instead of the color of the different density, the color may be changed according to the number of examinations, or the brightness of the cell may be changed according to the number of examinations.

In the present embodiment, the number of examinations is represented by the density of the color in the matrix. Therefore, the representation result shown in FIG. 6 is a representation in which the number of examinations in each time slot on each day of the week is represented as a heat map.

In the representation result 31, the size of the circle superimposed on each cell represents the number of medical workers. As described above, since the number of medical workers is classified into three ranges by the threshold value processing, three types of circles having different sizes are superimposed on each cell. In addition, the number of medical workers is displayed in the circle.

In the representation result 31, the color of the circle representing the number of medical workers represents the workload. For example, the workload is represented by red for a workload equal to or greater than the above-mentioned threshold value Th1 and by green for a workload less than the threshold value Th1. That is, the workload equal to or higher than the threshold value Th1 is emphasized by red. In a case where two or more threshold values are used, for example, the workload may be represented in three or more colors such as red, green, and blue. In FIG. 6, red is represented by a black circle, and green is represented by a white circle.

In the period area 32, the period of the action information displayed as the representation result 31 is displayed. FIG. 6 shows that the representation result 31 based on the action information for 183 days from Thursday, Jun. 29, 2023 to Monday, Dec. 18, 2023 is displayed.

In the first embodiment, in the period area 32, it is possible to designate any period for the action information represented by the representation result 31. For example, the calendar is displayed by selecting the period area 32, and any period can be designated by designating the start date and the end date in the calendar.

In a case where the period is designated, the first derivation unit 23 extracts the action information of the designated period from all the action information, and derives the numerical information and the number of medical workers related to the action of the medical worker for each of the plurality of groups based on the extracted action information. The second derivation unit 24 derives the workload based on the numerical information and the number of medical workers derived based on the action information of the designated period. The representation unit 25 derives the representation result 31 from the numerical information derived based on the action information for the designated period, the number of medical workers, and the workload, and displays the representation result 31 on the display 14.

The attribute information selection area 33 is a region for selecting the attribute information to be assigned to the horizontal axis of the representation result 31. In FIG. 6, in addition to the day of the week as the initial value, the technician, the examination type, the examination room, and the examination date are displayed to be selectable. The attribute information is not limited thereto. The rectangular shape 33A is assigned to the currently selected attribute information. The operator can select any attribute information displayed in the attribute information selection area 33 by using the input device 15.

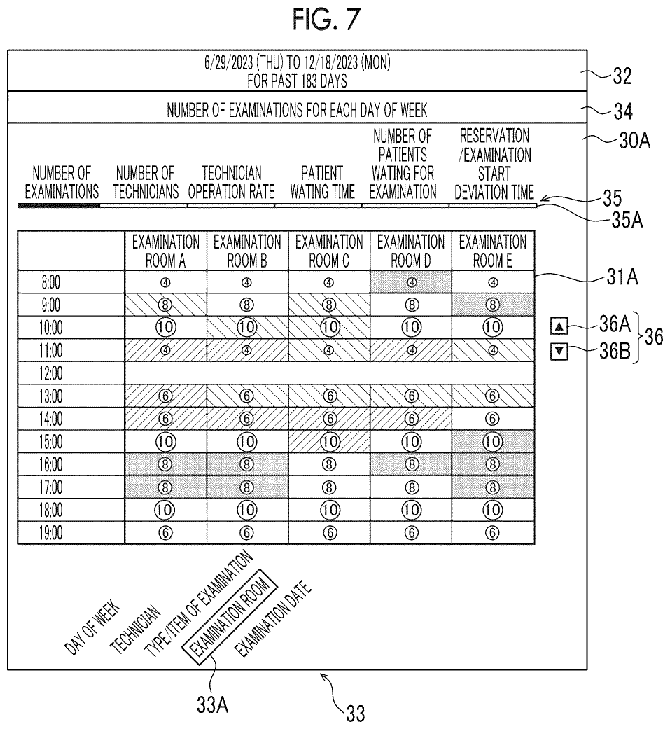

In a case where the attribute information is selected, the classification unit 22 classifies the action information into a plurality of groups based on the selected attribute information. For example, in a case where the examination room is selected instead of the day of the week, the action information is classified into a plurality of groups using two pieces of attribute information of the examination room and the time slot. The first derivation unit 23 derives the numerical information and the number of medical workers for each of the plurality of groups classified by using the selected attribute information. The second derivation unit 24 derives the workload based on the numerical information and the number of medical workers. The representation unit 25 derives a representation result 31 in which examination rooms are assigned to the horizontal axis instead of the day of the week. FIG. 7 is a diagram showing a display screen 30A that displays a representation result 31 in which examination rooms are assigned to a horizontal axis instead of days of the week. In FIG. 7, the representation result 31A for the five examination rooms A to E is shown.

In the title area 34, a title of the matter displayed in the representation result 31 is displayed. On the display screen 30 shown in FIG. 6, a title of “number of examinations for each day of the week” is displayed. In addition, a title of “number of examinations per examination room” is displayed in FIG. 7.

The numerical information selection area 35 is a region for selecting the numerical information represented by each cell of the matrix in the representation result 31. In FIG. 6, in addition to the number of examinations as the initial value, the number of technicians, the technician operation rate, the patient waiting time, the number of patients waiting for the examination, and the reservation/examination deviation time are displayed to be selectable. The operator can select any numerical information in the numerical information selection area 35 by using the input device 15. In FIG. 6, a bar 35A is displayed in the numerical information selection area 35, and the brightness of a portion corresponding to the numerical information currently selected in the bar 35A is lower than the brightness of other portions. Accordingly, it is possible to recognize which numerical information is selected in the displayed representation result 31.

In a case where the numerical information is selected, the first derivation unit 23 derives the selected numerical information and the number of medical workers for each of the plurality of groups. The second derivation unit 24 derives the workload based on the selected numerical information and the number of medical workers. The representation unit 25 derives a representation result 31 in which the selected numerical information is represented instead of the number of examinations. For example, in a case where the number of technicians is selected instead of the number of examinations, the representation unit 25 displays a display screen on which the representation result 31B in which colors having different densities are assigned to the cells according to the number of technicians is displayed. Even in this case, the difference in gray and hatching applied to the cell indicates the number of technicians. In addition, the title is changed to “number of technicians per day of week”.

An ascending sort button 36A and a descending sort button 36B are displayed in the sort area 36. The operator can select a horizontal axis or a vertical axis in the representation result 31 and select the ascending sort button 36A and the descending sort button 36B to rearrange the numerical information related to the attribute information assigned to the selected axis in ascending order or descending order. For example, in a case where the day of the week is assigned to the horizontal axis, in a case where the operator selects the horizontal axis and selects the ascending sort button 36A, the days of the week are rearranged in descending order of the total number of examinations in all time slots derived on each day of the week.

On the other hand, since the vertical axis is a time slot, in a case where the operator selects the vertical axis and selects the descending sort button 36B, the time slots are rearranged in descending order of the total number of the examination numbers derived in each time slot on all the days of the week.

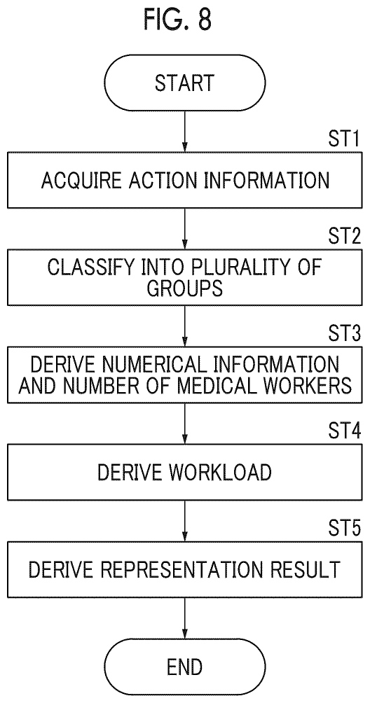

Next, a processing performed in the first embodiment will be described. FIG. 8 is a flowchart showing the processing performed in the first embodiment. For example, the processing is started by the operator giving an instruction to the medical service support device 1, and the information acquisition unit 21 acquires the action information from the management server 2 (step ST1). Next, the classification unit 22 classifies the action information into a plurality of groups based on the attribute information related to the classification (step ST2).

Subsequently, the first derivation unit 23 derives the numerical information and the number of medical workers related to the action of the medical worker based on the action information for each of the plurality of groups (step ST3). Next, the second derivation unit 24 derives the workload in each of the plurality of groups based on at least the numerical information (step ST4). Then, the representation unit 25 derives the representation result in which the numerical information, the number of medical workers, and the workload are represented in a unit of the classified group (step ST5), and the processing ends.

As described above, in the first embodiment, the action information related to the action of the medical worker is acquired, the action information is classified into a plurality of groups based on the attribute information related to the classification, the numerical information and the number of medical workers related to the action of the medical worker are derived for each of the plurality of groups based on the action information, and the representation result in which the numerical information and the number of medical workers are represented in units of the classified groups is derived. Therefore, the numerical information and the number of medical workers related to the action of the medical worker can be grasped simultaneously by the representation result.

In addition, in the first embodiment, since the representation result representing the workload is derived, the workload can be further grasped by referring to the representation result.

In addition, on the matrix, a representation result in which the numerical information and the number of medical workers are represented in a plurality of group units is derived. Therefore, it is possible to easily grasp the relationship between the attribute information, the numerical information, and the number of medical workers.

In the first embodiment, the number of examinations, the number of medical workers, and the workload are represented as a heat map in a plurality of group units on a matrix defined by a horizontal axis to which a day of the week is assigned and a vertical axis to which a time slot is assigned, but the present invention is not limited thereto. For example, the action information for all the examinations performed in the hospital may be acquired, and a representation result in which the number of examinations and the medical worker for each examination type are represented as a tree map may be derived. This will be described below as a second embodiment.

The functional configuration of the medical service support device according to the second embodiment is the same as the functional configuration of the medical service support device according to the first embodiment, and only the processing to be performed is different. Therefore, the detailed description of the configuration of the device will be omitted here.

In the second embodiment, the classification unit 22 uses two pieces of attribute information of the examination type and the examination item to classify the action information into a plurality of groups for each examination type and each examination item. Here, the examination item is included in the examination type. Therefore, the classification unit 22 classifies the action information into a plurality of groups for each examination type, and classifies the action information for each examination item in the classified group for each examination type.

In the second embodiment, for example, five types of examinations such as an ultrasound examination, an electrocardiogram examination, an electromyogram examination, a respiratory function examination, and other examinations are used as the examination type. As examination items of the ultrasound examination, for example, abdominal ultrasound examination, cardiac ultrasound examination, body surface ultrasound examination, carotid ultrasound examination, lower limb ultrasound examination, and other ultrasound examinations are used. As the examination items of the electrocardiogram examination, for example, a 12-lead electrocardiogram, a Holter electrocardiogram, a treadmill, and other electrocardiogram examinations are used.

As the examination items of the electromyogram, for example, auditory evoked potentials (AEP), somatosensory evoked potentials (SEP), and visual evoked potentials (VEP) are used. Hereinafter, the auditory brainstem response, the somatosensory evoked potential, and the visual evoked potential are referred to as AEP, SEP, and VEP, respectively.

As the respiratory function test, preoperative (vital capacity (VC)) and forced vital capacity (FVC), ventilation (vital capacity (VC)) and forced vital capacity (FVC), functional residual capacity (FRC), and basal metabolism are used. The preoperative and forced vital capacity, the ventilation and the forced vital capacity, and the functional residual capacity are respectively referred to as VC+FVC and VC+FVC+FRC. As the examination items of other examination types, an electroencephalogram examination and a hearing examination are used.

The first derivation unit 23 derives the numerical information and the number of medical workers for each of a plurality of groups, that is, for each examination type. Further, the first derivation unit 23 derives the numerical information and the number of medical workers for each examination item. The first derivation unit 23 derives the number of examinations and the number of medical workers within the period included in the action information. In the second embodiment, the second derivation unit 24 does not derive the workload.

The representation unit 25 derives a representation result in which the number of examinations and the number of medical workers are represented as a tree map in a unit of the classified group. In the second embodiment, the representation unit 25 first divides the basic region having a predetermined size into regions having sizes corresponding to the number of examinations for each examination type. In the second embodiment, since five types of examination types are used, the representation unit 25 divides the basic region into five regions having sizes corresponding to the number of examinations. Since the basic region has a predetermined size, the five regions have relative sizes according to the number of examinations.

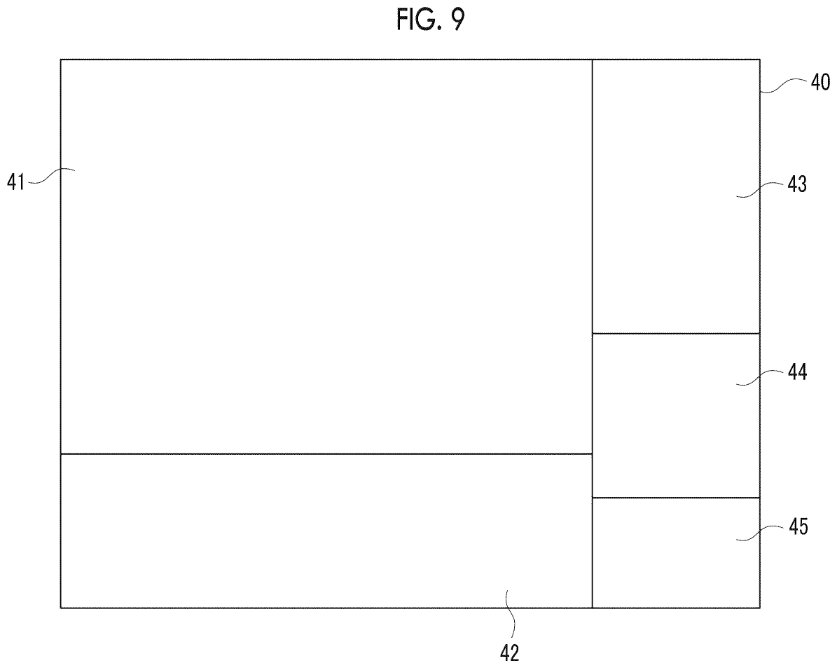

FIG. 9 is a diagram showing a basic region divided into regions having sizes corresponding to the number of examinations of the examination type in the second embodiment. As shown in FIG. 9, the basic region 40 is divided into a first region 41 representing the number of examinations of the ultrasound examination, a second region 42 representing the number of examinations of the electrocardiogram examination, a third region 43 representing the number of examinations of the electromyogram examination, a fourth region 44 representing the number of examinations of the respiratory function examination, and a fifth region 45 representing the number of examinations of other examinations. As shown in FIG. 9, the size of the region is reduced in the order of the first region 41, the second region 42, the third region 43, the fourth region 44, and the fifth region 45. Therefore, in a case where the size of the divided region is viewed, it is found that the number of examinations of the ultrasound examination is the largest, and the number of examinations of the electrocardiogram examination, the electromyogram examination, the respiratory function examination, and other examinations is decreased in this order.

The representation unit 25 further divides each of the first region 41, the second region 42, the third region 43, the fourth region 44, and the fifth region 45, which are further divided, into regions having a relative size corresponding to the number of examinations of the examination item.

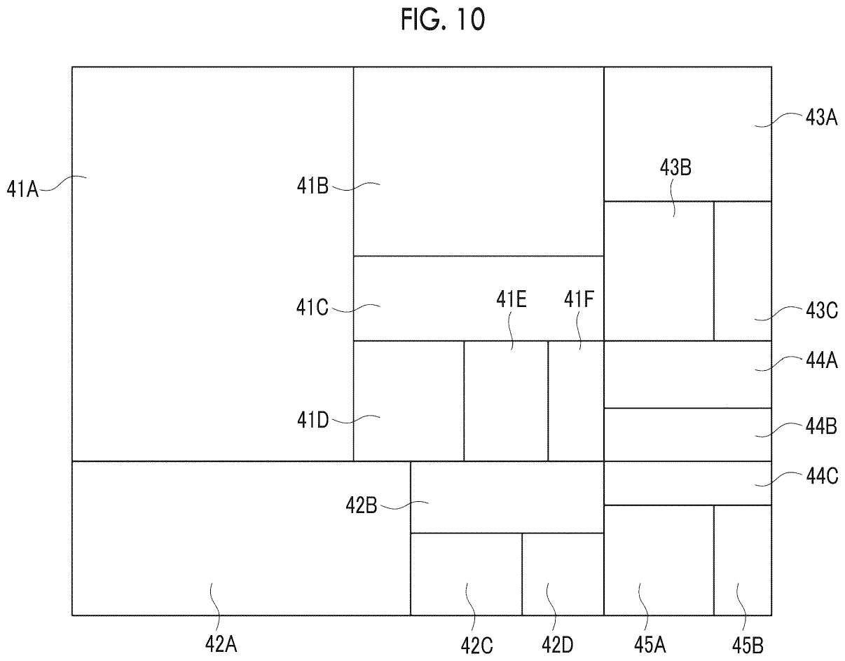

FIG. 10 is a diagram showing a basic region further divided into regions having sizes corresponding to the number of examinations of the examination items. As shown in FIG. 10, the first region 41 is divided into a region 41A representing the number of abdominal ultrasound examinations, a region 41B representing the number of cardiac ultrasound examinations, a region 41C representing the number of body surface ultrasound examinations, a region 41D representing the number of carotid ultrasound examinations, a region 41E representing the number of lower limb ultrasound examinations, and a region 41F representing the number of other ultrasound examinations. As shown in FIG. 10, the sizes of the regions 41A to 41F in the first region 41 are reduced in this order. Therefore, in a case where the sizes of the regions 41A to 41F in the first region 41 are viewed, it is found that the number of examinations of the abdominal ultrasound examination is the largest, and the number of examinations of the cardiac ultrasound examination, the body surface ultrasound examination, the carotid ultrasound examination, the lower limb ultrasound examination, and other ultrasound examinations is decreased in this order.

The second region 42 is divided into a region 42A representing the number of examinations of the 12-lead electrocardiogram, a region 42B representing the number of examinations of the Holter electrocardiogram, a region 42C representing the number of examinations of the treadmill examination, and a region 42D representing other electrocardiogram examinations. As shown in FIG. 10, the sizes of the regions 42A to 42D in the second region 42 are smaller in this order. Therefore, in a case where the sizes of the regions 42A to 42D in the second region 42 are viewed, it is found that the number of examinations of the 12-lead electrocardiogram is the largest, and the number of examinations of the Holter electrocardiogram examination, the treadmill examination, and other electrocardiogram examinations is decreased in this order.

The third region 43 is divided into a region 43A representing the number of ABR examinations, a region 43B representing the number of SEP examinations, and a region 43C representing the number of VEP examinations. As shown in FIG. 10, the sizes of the regions 43A to 43C in the third region 43 are smaller in this order. Therefore, in a case where the sizes of the regions 43A to 43C in the third region 43 are viewed, it is found that the number of examinations of the ABR examination is the largest, and the number of examinations of the SEP examination and the VEP examination is decreased in this order.

The fourth region 44 is divided into a region 44A representing the number of examinations of the VC+FVC examination, a region 44B representing the number of examinations of the VC+FVC+FRC examination, and a region 44C representing the number of examinations of the basal metabolism examination. As shown in FIG. 10, the sizes of the regions 44A to 44C in the fourth region 44 are smaller in this order. Therefore, in a case where the sizes of the regions 44A to 44C in the fourth region 44 are viewed, it is found that the number of examinations of the VC+FVC examination is the largest, and the number of examinations of the VC+FVC+FRC examination and the basal metabolism examination is decreased in this order.

The fifth region 45 is divided into a region 45A representing the number of examinations of the electroencephalogram examination and a region 45B representing the number of examinations of the hearing examination. As shown in FIG. 10, the sizes of the regions 45A and 45B in the fifth region 45 are smaller in this order. Therefore, in a case where the sizes of the regions 45A and 45B in the fifth region 45 are viewed, it is found that the number of examinations of the electroencephalogram examination is larger than the number of examinations of the hearing examination.

In the second embodiment, the representation unit 25 represents the number of medical workers for each group in the region divided into the group for each examination item. Specifically, the number of medical workers is represented by superimposing the same number of human-shaped marks as the number of medical workers in the region.

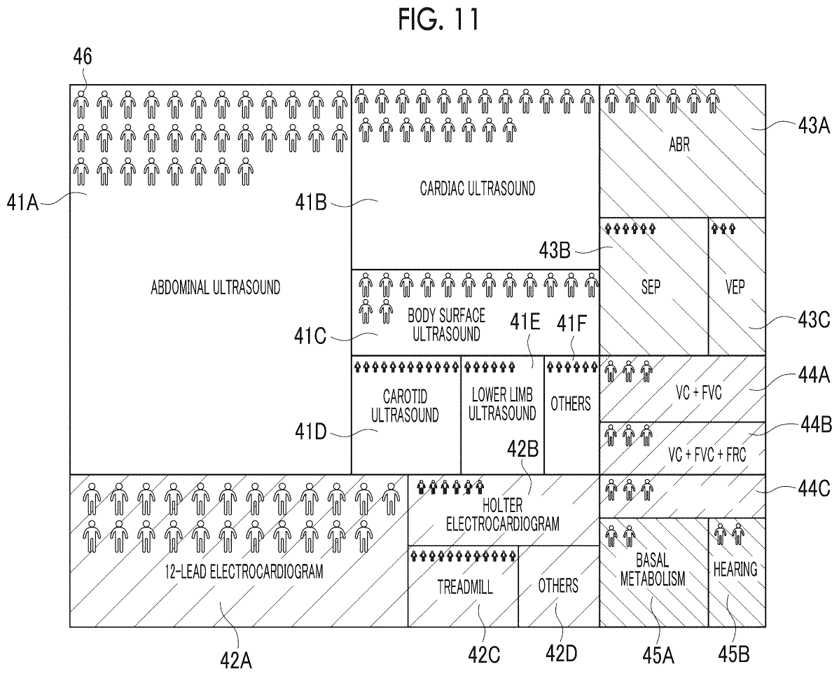

Then, the representation unit 25 assigns different colors to the examination types, derives a representation result in which the examination item is given to the examination item by the text, and displays the representation result on the display 14. FIG. 11 is a diagram showing a representation result derived in the second embodiment. As shown in FIG. 11, for example, blue is assigned to an ultrasound examination, red is assigned to an electrocardiogram examination, green is assigned to an electromyogram examination, yellow is assigned to a respiratory function examination, and gray is assigned to other examinations. In FIG. 11, different hatching indicates that the colors are different. Instead of different colors, colors having different densities may be assigned. In addition, the examination item name is superimposed on the region for each examination item. In addition, as shown in FIG. 11, a human-shaped mark 46 of the same number as the medical workers is superimposed on the region for each medical item. The mark 46 is not limited to a human shape and may be a circular shape, a triangular shape, a quadrangular shape, or the like. In addition, the same number of icons as the number of medical workers may be arranged, and the number of medical workers may be represented by an icon representing a single person and an icon representing a plurality of persons (for example, 10 persons). For example, in a case where an icon representing one person is represented by ∘ and an icon representing ten persons is represented by •, four persons may be represented by ∘∘∘∘ and thirteen persons may be represented by •∘∘∘.

As in the second embodiment, the numerical information related to the action of the medical worker and the number of medical workers can be grasped simultaneously by deriving the representation result by the tree map.

In addition, in the first embodiment, the icon may be used as in the second embodiment to represent the number of examinations or the number of medical workers. In addition, in the second embodiment, a numerical value may be used instead of the icon to represent the number of medical workers, as in the first embodiment. In addition, in the first embodiment, the medical worker may be indicated by a heat map, and the number of examinations may be indicated by a numerical value. In addition, in the second embodiment, the area may be defined according to the medical worker, and the number of examinations may be indicated by an icon.

In addition, in the first embodiment, the second derivation unit 24 derives the workload, but the present disclosure is not limited thereto. The second derivation unit 24 may be omitted from the medical service support device according to the first embodiment, and the workload may not be derived. In this case, the color of the circle representing the number of medical workers may be the same color.

In addition, in the first and second embodiments, the medical service support device may be a system consisting of a plurality of devices. For example, a device that performs the processing of the information acquisition unit 21, the classification unit 22, the first derivation unit 23, and the second derivation unit 24 and a device that performs the processing of the representation unit 25 may be separately provided.

In this embodiment, each process is executed on an arbitrary computer. The arbitrary computer may execute these processes by means of a processor as hardware, a program as software, or a combination of the processor and the program. In such a case, the processor is configured to execute the various processes in this embodiment in cooperation with the program and may function as each unit or means in this embodiment. In addition, the order in which the processor executes these processes is not limited to the order described in this embodiment and may be changed as appropriate. The arbitrary computer may be a general-purpose computer, a computer for a specific purpose, a workstation, or any other system capable of executing each process.

The processor may be configured by one or more hardware, and the type of hardware is not limited. For example, the processor may comprise at least one of programmable logic devices such as CPUs (Central Processing Units), MPUs (Micro Processing Units), and FPGAs (Field Programmable Gate Arrays); dedicated circuits for performing specific processes such as ASICs (Application Specific Integrated Circuits); and other hardware such as a GPU (Graphics Processing Unit) and an NPU (Neural Processing Unit). The hardware may also be a combination of different types of hardware. When multiple hardware are configured to execute one or more processes of a processor, the said multiple hardware may exist in devices that are physically separate from each other, or in the same device. In any embodiment, the order of each process by the processor is not limited to the order described above and may be changed as appropriate. The hardware is configured by an electric circuit (circuitry) etc. that combines circuit elements such as semiconductor devices.

Furthermore, the program may be firmware or software such as microcode. The program may also be a group of program modules, each function of which may be performed by a processor configured to execute each of the program modules. The program may be program code or code segments stored on one or more non-transitory computer-readable media (e.g., storage media or other storage). The program may be stored in separate non-transitory computer-readable media located on devices that are physically separate from each other. The program code or code segments may represent any combination of procedures, functions, subprograms, routines, subroutines, modules, software packages, classes, instructions, data structures, or program statements. The program code or code segments may be connected to other code segments or hardware circuits by sending or receiving information, data, arguments, parameters, or memory contents.

In the above embodiment, it is explained that the medical service support program 12 is stored (installed) in advance in the storage 13, but this is not limited to this. The medical service support program 12 may be provided in a form recorded on a recording medium such as a CD-ROM (Compact Disc Read Only Memory), DVD-ROM (Digital Versatile Disc Read Only Memory), and USB (Universal Serial Bus) memory. In addition, the medical service support program 12 may be provided in a form that the medical service support program 12 are downloaded from an external device via a network.

The technology of this disclosure also extends to all types of program products. Program products include all types of products for providing programs. For example, program products include programs provided via networks such as the Internet, and non-temporary computer readable storage media such as CD-ROMs, DVDs, and USB memory devices that store programs.

The supplementary notes of the present disclosure will be described below.

### Supplementary Note 1

A medical service support device comprising:
a processor,
wherein the processor is configured to:
   acquire action information related to an action of a medical worker;
   classify the action information into a plurality of groups based on attribute information related to classification;
   derive numerical information related to the action of the medical worker and the number of medical workers based on the action information for each of the plurality of groups; and
   derive a representation result in which the numerical information and the number of medical workers are represented in a unit of the classified group.

### Supplementary Note 2

The medical service support device according to Supplementary Note 1,
wherein the processor is configured to classify the action information into a plurality of groups based on designated attribute information among a plurality of pieces of attribute information.

### Supplementary Note 3

The medical service support device according to Supplementary Note 1 or 2,
wherein the processor is configured to derive designated numerical information among a plurality of pieces of numerical information.

### Supplementary Note 4

The medical service support device according to any one of Supplementary Notes 1 to 3,
wherein the processor is configured to:
   extract the action information according to a designated condition; and
   derive the numerical information and the number of medical workers based on the extracted action information.

### Supplementary Note 5

The medical service support device according to any one of Supplementary Notes 1 to 4,
wherein the attribute information includes first attribute information and second attribute information, and
the processor is configured to derive a representation result in which the numerical information and the number of medical workers are represented for each of the plurality of groups on a matrix defined by a first axis to which the first attribute information is assigned and a second axis to which the second attribute information is assigned.

### Supplementary Note 6

The medical service support device according to Supplementary Note 5,
wherein the processor is configured to sort the plurality of groups with respect to at least one of the first axis or the second axis based on the numerical information or the number of medical workers.

### Supplementary Note 7

The medical service support device according to any one of Supplementary Notes 1 to 6,
wherein the attribute information is at least one of a time slot, a day of a week, a medical worker who performed the action, an action performance date, a room in which the action was performed, a device used for the action, or an action type.

### Supplementary Note 8

The medical service support device according to Supplementary Note 7,
wherein the processor is configured to derive a representation result representing any one of the numerical information or the number of medical workers in each of the plurality of groups by regions in which at least one of a color, a density, or a size differs depending on the number of any one of the numerical information or the number of medical workers.

### Supplementary Note 9

The medical service support device according to Supplementary Note 8,
wherein the processor is configured to superimpose an indicator that indicates the number of the other of the numerical information and the number of medical workers on the region to derive a representation result representing the other of the numerical information or the number of medical workers.

### Supplementary Note 10

The medical service support device according to any one of Supplementary Notes 1 to 9,
wherein the processor is configured to:
   derive a workload in each of the plurality of groups based on at least the numerical information; and
   derive the representation result representing the workload in each of the plurality of groups.

### Supplementary Note 11

The medical service support device according to Supplementary Note 10,
wherein the processor is configured to determine the workload based on one or more predetermined threshold values to be emphasized, and derive the representation result in which the determined workload is emphasized.

### Supplementary Note 12

A medical service support method comprising:
causing a computer to be configured to:
   acquire action information related to an action of a medical worker;
   classify the action information into a plurality of groups based on attribute information related to classification;
   derive numerical information related to the action of the medical worker and the number of medical workers based on the action information for each of the plurality of groups; and
   derive a representation result in which the numerical information and the number of medical workers are represented in a unit of the classified group.

### Supplementary Note 13

A medical service support program causing a computer to execute:
a procedure of acquiring action information related to an action of a medical worker;
a procedure of classifying the action information into a plurality of groups based on attribute information related to classification;
a procedure of deriving numerical information related to the action of the medical worker and the number of medical workers based on the action information for each of the plurality of groups; and
a procedure of deriving a representation result in which the numerical information and the number of medical workers are represented in a unit of the classified group.

What is claimed is:

1. A medical service support device comprising:
a processor,
wherein the processor is configured to:
acquire action information related to an action of a medical worker;
classify the action information into a plurality of groups based on attribute information related to classification;
derive numerical information related to the action of the medical worker and the number of medical workers based on the action information for each of the plurality of groups; and
derive a representation result in which the numerical information and the number of medical workers are represented in a unit of the classified group.

2. The medical service support device according to claim 1,
wherein the processor is configured to classify the action information into a plurality of groups based on designated attribute information among a plurality of pieces of attribute information.

3. The medical service support device according to claim 1,
wherein the processor is configured to derive designated numerical information among a plurality of pieces of numerical information.

4. The medical service support device according to claim 1,
wherein the processor is configured to:
extract the action information according to a designated condition; and
derive the numerical information and the number of medical workers based on the extracted action information.

5. The medical service support device according to claim 1,
wherein the attribute information includes first attribute information and second attribute information, and
the processor is configured to derive a representation result in which the numerical information and the number of medical workers are represented for each of the plurality of groups on a matrix defined by a first axis to which the first attribute information is assigned and a second axis to which the second attribute information is assigned.

6. The medical service support device according to claim 5,
wherein the processor is configured to sort the plurality of groups with respect to at least one of the first axis or the second axis based on the numerical information or the number of medical workers.

7. The medical service support device according to claim 1,
wherein the attribute information is at least one of a time slot, a day of a week, a medical worker who performed the action, an action performance date, a room in which the action was performed, a device used for the action, or an action type.

8. The medical service support device according to claim 7,
wherein the processor is configured to derive a representation result representing any one of the numerical information or the number of medical workers in each of the plurality of groups by regions in which at least one of a color, a density, or a size differs depending on the number of any one of the numerical information or the number of medical workers.

9. The medical service support device according to claim 8,
wherein the processor is configured to superimpose an indicator that indicates the number of the other of the numerical information or the number of medical workers on the region to derive a representation result representing the other of the numerical information or the number of medical workers.

10. The medical service support device according to claim 1,
wherein the processor is configured to:
derive a workload in each of the plurality of groups based on at least the numerical information; and
derive the representation result representing the workload in each of the plurality of groups.

11. The medical service support device according to claim 10,
wherein the processor is configured to determine the workload based on one or more predetermined threshold values to be emphasized, and derive the representation result in which the determined workload is emphasized.

12. A medical service support method comprising:
causing a computer to be configured to:
acquire action information related to an action of a medical worker;
classify the action information into a plurality of groups based on attribute information related to classification;
derive numerical information related to the action of the medical worker and the number of medical workers based on the action information for each of the plurality of groups; and
derive a representation result in which the numerical information and the number of medical workers are represented in a unit of the classified group.

13. A non-transitory computer-readable storage medium that stores a medical service support program causing a computer to execute:
a procedure of acquiring action information related to an action of a medical worker;
a procedure of classifying the action information into a plurality of groups based on attribute information related to classification;
a procedure of deriving numerical information related to the action of the medical worker and the number of medical workers based on the action information for each of the plurality of groups; and
a procedure of deriving a representation result in which the numerical information and the number of medical workers are represented in a unit of the classified group.
